(19) United States
(12) Patent Application Publication
(10) Pub. No.: US 2020/0377596 A1
(43) Pub. Date: Dec. 3, 2020

(54) DOSING OF BISPECIFIC T CELL ENGAGER

(71) Applicant: Amphivena Therapeutics Inc., South San Francisco, CA (US)

(72) Inventor: Victoria Smith, South San Francisco, CA (US)

(21) Appl. No.: 16/888,427

(22) Filed: May 29, 2020

Related U.S. Application Data
(60) Provisional application No. 62/931,742, filed on Nov. 6, 2019.
(60) Provisional application No. 62/853,802, filed on May 29, 2019.

(51) Int. Cl. C07K 16/28

(52) U.S. Cl. CPC C07K 16/2809; A61K 9/0019; C07K 2317/31; C07K 2317/622

(57) ABSTRACT

Methods for reducing myeloid-derived suppressor cells and activating T cells in a patient and for treating a patient suffering from a solid tumor are described. The methods entail administering a CD3/CD33 T cell engager.

### SEQUENCE LISTING

The instant application contains a Sequence Listing which has been submitted electronically in ASCII format and is hereby incorporated by reference in its entirety. Said ASCII copy, created on Jun. 17, 2020, is named Sequence Listing and is 4.46 KB in size.

### BACKGROUND

T cell engagers, a particular class of bispecific antibody, mediate binding between a target cell and a T cell resulting in T cell directed lysis and T cell activation, differentiation and proliferation. While T cell engagers have demonstrated impressive potency and anti-tumor activity in some settings, a barrier to broader therapeutic success in many cases is undesirable activity against normal cells expressing the target of interest. This “on target, off tumor” toxicity can be significant, and has been reported widely for engineered T cell approaches.

Myeloid derived suppressor cells (MDSC) act locally and systemically to impair anti-tumor immunity, inhibiting effector T cell responses, promoting formation of immune suppressive regulatory T cells, inhibiting the maturation of dendritic cells and antigen presentation, and promoting formation of metastases. MDSC elicit a range of suppressive functions that inhibit normal T cell responses as well as causing unresponsiveness to immune checkpoint blockade. A dominant function of MDSC is the suppression of T cell activity in a variety of manners that are pathology and context dependent. The presence of MDSC is thought to be associated with poor outcomes and lack of response to certain therapies, e.g., therapies that activate T cells and therapies involving the use of a checkpoint inhibitor. Moreover, compensatory myelopoiesis in response to T cell activation can result in further generation of MDSC and recruitment to tumors.

### SUMMARY

Described herein are methods for using AMV564, a bispecific, bivalent molecule which binds to CD3 and CD33. AMV564 is homodimeric protein (i.e., a homodimer of a polypeptide having the amino acid sequence of SEQ ID NO: 1) having four single-chain variable fragment (scFv) binding sites, two that bind CD33 and two that bind CD3. A bivalent design can, in theory, restore selectivity to a T cell engager, directing preferential binding to regions of high local target density, such as found at sites of active signaling or associated with high receptor density or expression. Despite the fact that AMV564 binds CD33, which is broadly expressed across the myeloid lineage, it can be dosed in a manner that that provides a desirable therapeutic index, with selective binding of MDSC. AMV564 is very selective over a wide dose range, without being bound by any particular theory, this may be due to some combination of: bivalency, the affinity of the scFv and the geometry of the homodimer. For example, again without being bound by any particular theory, the structure of AMV564 may allow it to bind clusters of dimerized CD33.

AMV564 has dual activity: it induces T cell mediated killing of MDSC and drives T cell activation, promoting favorable polarization (e.g., Th1 CD4 T cells and effector CD8 T cells). AMV564 has an EC50 for MDSC that is less than about 3 pM. Dosed appropriately, AMV564 largely spares neutrophils, monocytes and many differentiated myeloid cells while directing killing of MDSC, thereby inhibiting MDSC-suppressive pathways.

Peripheral MDSC may play important roles in T cell suppression and T cell trafficking to tumor sites, a potentially rate-limiting factor for T cell activation-based therapies. For example, a 15 mcg dose of AMV564 can, in some circumstance, achieve depletion of MDSC populations. As MDSC are recruited from the bone marrow, peripheral depletion may enable sufficient control to benefit anti-tumor immunity over a dosing timeframe that exceeds the limited longevity of tissue-resident MDSC. However, distribution of AMV564 in the tumor microenvironment could target MDSC at tumor sites while promoting expansion of local T cells. Moreover, AMV564 delivery or entry into draining lymph nodes in addition to the periphery, for example, doses of up to 50, 75 and 100 mcg by CIV route, could achieve de-repression of anti-tumor T cells and restore antigen presentation and immune homeostasis. Subcutaneous delivery of AMV564, for example at doses of 5, 15, or 50 mcg, provides a direct mechanism of initial distribution in the lymphatic system, including tumor draining lymph nodes. With subcutaneous administration AMV564 is effective at lower doses, likely due to the access to the lymph system.

AMV564 can both relieve immune suppression and activate T cell effector function in cancer patients. AMV564 can do so by relieving immunosuppression via targeted depletion of myeloid derived suppressor cells (MDSC) and by directly activating/repolarizing T cells and improved T effector function.

Importantly, subcutaneous administration of AMV564 facilitates immune activation by targeting the lymphatic system.

In one aspect of the methods of treatment described herein the target cell is a leukemic blast, and a dosing regimen that establishes a steady-state exposure of 30-70 pM AMV564 is used to reduce leukemic blasts in AML, Myelodysplastic Syndrome (MDS) and other cancers with high CD33 expression. Such a regimen could be achieved by dosing at 50-125 mcg (“micrograms”) by continuous intravenous infusion (CIV), for example, 50 mcg, 75 mcg, 100 mcg.

Also described herein is a method of for controlling both MDSC by administering 5-125 mcg (for example, 50 mcg, 75 mcg, 100 mcg) of AMV564 by intravenous infusion. In some cases, for example, MDS patients with a lower blast burden MDSC likely play a more critical role in disease progression. In such cases, a 5, 15, 30, 50 mcg AMV564 can be administered, e.g., by intravenous infusion.

Described herein is method for reducing myeloid-derived suppressor cells and activating T cells in a patient, the method comprising administering AMV564 (a polypeptide having the amino acid sequence of SEQ ID NO: 1) to the patient. In various embodiments: AMV564 is administered by subcutaneous injection; the dose of AMV564 injected is 5-150 mcg (micrograms); the AMV564 is administered on a least 7 days (8, 9, 10, 11, 12, 13 or 14 days) over a 14 day period; the AMV564 is administered daily (e.g., at 5, 10, 15, 20, 25, 30, 35, 40, 45, 50, 55, 60, 65, 70, 75, 80, 85, 90, 95, 100, 105, 110, 115, 120, 125, 130, 135, 140, 145, or 150 mcg/day) subcutaneously; the AMV6564 is administered on 10 days over a 14 day period; the AMV6564 is administered on 5 consecutive days on two occasions over a 14 day period; the AMV6564 is administered on 5 consecutive days, is not administered on the following two days and is administered on the following 5 consecutive days; the AMV564 is administered over a 21 day cycle in which AMV564 is administered on at least 7 days over a 14 day period and is not administered over the subsequent 7 day period; the 21 day cycle is repeated at least two times; AMV564 is administered on at least 10 days over a 14 day period with administration on 5 consecutive days followed by 2 days of no administration followed by administration on 5 consecutive days; the dose of AMV564 administered is 5, 10, 15, 20, 25, 30, 35, 40, 45, 50, 55, 60, 65, 70, 75, 80, 85, 90, 95, 100, 105, 110, 115, 120, 125, 130, 135, 140, 145, or 150 mcg on each day when administered; the patient is being treated with a therapy that activates T cells (e.g., the therapy is a CAR T cell therapy; the therapy is a CTL therapy; the therapy is an antibody therapy; the therapy is treatment with a T cell engager that comprises a CD3 binding domain and activates T cells); the patient is suffering from or being treated for being treated for a leukemia (acute myeloid leukemia or myelodysplastic syndrome); the patient is suffering or being treated for a solid tumor; the solid tumor is selected from the group consisting of: pancreatic cancer, ovarian cancer, colon cancer, rectal cancer, non-small cell lung carcinoma, urothelial cancer, squamous cell carcinoma, rectal cancer, penile cancer, endometrial cancer, small bowel cancer, cancer of the appendix; the administration of AMV564 achieves a steady-state exposure of 30-70 pM AMV564.

Also described is a method for treating a patient suffering from a solid tumor, the method comprising administering AMV564 a polypeptide having the amino acid sequence of SEQ ID NO: 1) to the patient. In various embodiments: AMV564 is administered by subcutaneous injection; the dose of AMV564 injected is 5-150 mcg (micrograms); the AMV564 is administered on a least 7 days (8, 9, 10, 11, 12, 13 or 14 days) over a 14 day period; the AMV564 is administered daily (e.g., at 5, 10, 15, 20, 25, 30, 35, 40, 45, 50, 55, 60, 65, 70, 75, 80, 85, 90, 95, 100, 105, 110, 115, 120, 125, 130, 135, 140, 145, or 150 mcg/day) subcutaneously; the AMV6564 is administered on 10 days over a 14 day period; the AMV6564 is administered on 5 consecutive days on two occasions over a 14 day period; the AMV6564 is administered on 5 consecutive days, is not administered on the following two days and is administered on the following 5 consecutive days; the AMV564 is administered over a 21 day cycle in which AMV564 is administered on at least 7 days over a 14 day period and is not administered over the subsequent 7 day period; the 21 day cycle is repeated at least two times; AMV564 is administered on at least 10 days over a 14 day period with administration on 5 consecutive days followed by 2 days of no administration followed by administration on 5 consecutive days; the dose of AMV564 administered is 5, 10, 15, 20, 25, 30, 35, 40, 45, 50, 55, 60, 65, 70, 75, 80, 85, 90, 95, 100, 105, 110, 115, 120, 125, 130, 135, 140, 145, r 150 mcg on each day when administered; the patient is being treated with a therapy that activates T cells (e.g., the therapy is a CAR T cell therapy; the therapy is a CTL therapy; the therapy is an antibody therapy; the therapy is treatment with a T cell engager that comprises a CD3 binding domain and activates T cells); the patient is suffering from or being treated for being treated for a leukemia (acute myeloid leukemia or myelodysplastic syndrome); the patient is suffering or being treated for a solid tumor; the solid tumor is selected from the group consisting of: pancreatic cancer, ovarian cancer, colon cancer, rectal cancer, non-small cell lung carcinoma, urothelial cancer, squamous cell carcinoma, rectal cancer, penile cancer, endometrial cancer, small bowel cancer, cancer of the appendix; the administration of AMV564 achieves a steady-state exposure of 30-70 pM AMV564; the solid tumor is selected from the group consisting of: Small Cell Lung Cancer (NSCLC) (e.g., metastatic nonsquamous NSCLC, III NSCLC, metastatic NSCLC expressing PD-L1), melanoma, Merkel cell, Microsatellite Instability-High Cancer (e.g., unresectable or metastatic, microsatellite instability-high (MSI-H) or mismatch repair deficient); the patient has progressed on checkpoint blockade; the administration of AMV564 achieves a steady-state exposure of 30-70 pM AMV564; the method comprises administering 50-125 mcg AMV564 by continuous intravenous infusion.

Also descried is a method for treating AML by administering AMV564 to reach a steady-state exposure of 30-70 pM AMV564.

CD33, also known as Siglec-3, is transmembrane protein is expressed on cells of myeloid lineage. CD33 has long been regarded as an appealing target for acute myeloid leukemia (AML), due to both high prevalence and high expression on leukemic blasts. The function of CD33 is not well understood, but activation of CD33 signaling on early lineage myeloid cells, such as immune-suppressive myeloid-derived suppressor cells (MDSC), has been shown to result in expansion of MDSC and production of suppressive cytokines and factors. CD33 expression is used as one component of cell surface marker sets to identify these immune-suppressive monocytic and granulocytic cells (for example, by use of flow cytometry for cellular immune phenotyping). It is unclear what role, if any, that CD33 plays on more differentiated myeloid lineage cells such as mature monocytes, neutrophils, macrophages and dendritic cells. Indeed, knockout of CD33 in human cells using CRISPR technology has demonstrated that CD33 is not required for lineage differentiation, but it nonetheless continues to be expressed at varying levels on most of these cells, rendering it as a challenging target for a non-selective T cell engager, with respect to both safety and efficacy.

T cell engagers function by acting as a bridge between an antigen on a target cell and different antigen on a T cell, forming a ternary complex between the drug and two different cell types, thereby mimicking the formation of a natural T cell-target cell synapse that is generated in the course of an adaptive immune response. It is thought that between 10-100 molecules of drug must be appropriately bound in order to activate T cell killing, and T cells must be available also. This active state thus brings additional considerations for receptor occupancy and dosing beyond a standard model of, for example, inhibiting a ligand or receptor via biologic drug binding, where dosing strategies aim for maximum target coverage until unacceptable toxicity is reached. There are yet more factors to consider for T cell engagers that target a broadly expressed antigen, such as CD33. Apart from the potential safety risks associated with broad depletion of the myeloid lineage cells (which confer valuable protection against infection), this cellular population can be very large (e.g. there are up to 100 billion neutrophils generated daily in a human body) and efficacy could thus be compromised due to insufficiently suitable drug distribution and an inadequate supply of T cells to accomplish killing. The targeting of other populations of cells such as leukemic blasts or the even rarer immune-suppressive MDSC could thus be hampered by a broad distribution of drug across CD33-positive normal myeloid cells and the need for sufficient associated T cells to achieve killing. For these reasons, determine the appropriate dose, dose schedule and routed of administration for a T cell engager is unusually challenging, far more so than for monovalent, monospecific agents.

Binding Properties of AMV564 and Dose Selection

The bivalent design of AMV564 is reflected in its physical properties. AMV564 is very potent and demonstrates cellular killing at low receptor occupancy, with elimination of CD33 target cells demonstrated ex vivo or in vitro with picomolar or sub-picomolar EC50 values. AMV564 is a potent agonist, which can elicit biological activity at low receptor occupancy or low target binding levels. Binding studies using flow cytometry show that there is no binding of neutrophils, polymorphonuclear (PMN) leukocytes or monocytes at 1 or 10 pM AMV564 as compared to MDSC and leukemic blast cell line KG1, which are both potently bound at 1 and 10 pM (FIGS. 1F-1G). At concentrations of 1 and 10 pM, AMV564 is thus highly selective for MDSC as compared to other abundant CD33 expressing cells The

Thus, an optimal therapeutic window for both safety and anti-tumor (leukemic blasts) and/or anti-suppressor (MDSC) cell activity might be obtained when engagement of target cells by AMV564 is sufficient for activity, but binding of other myeloid cells is minimal. Apart from safety considerations, excessive engagement of the large population of cells encompassing the normal myeloid lineage could result in reduction in efficacy due to suboptimal drug distribution and inadequate available T cells to achieve formation of the necessary ternary complexes that mimic a natural T cell synapse to facilitate cell killing.

Depletion of MDSC and Dose Selection

Overcoming the suppressive tumor microenvironment is a major challenge in immune therapy. The critical cellular effectors of the suppressive tumor microenvironment are MDSC, which are associated with immune dysfunction, repression of anti-tumor immunity and poor response to immunotherapy. MDSC suppress T cell and NK cell responses via a variety of cytokines, active species and pathways. In addition, they repress effective antigen presentation by dendritic cells in tumor draining lymph nodes. In another aspect of this disclosure, AMV564 depletes MDSC in the periphery and bone marrow of AML patients at low doses. The rapid decline observed at low, lead-in doses of AMV546 indicates potent binding and depletion of both monocytic and granulocytic MDSC populations. MDSC are rare in the periphery of a healthy adult and become significantly elevated in cancer patients. However, they are nonetheless relatively rare cells as compared to the mature myeloid lineage in general, and the efficacy of their depletion and control could be reduced at higher doses when overall receptor occupancy is unfavorable for selectivity for a bivalent T cell engager such as AMV564.

Depletion of MDSC in addition to leukemic blasts is beneficial in AML with respect to maintaining T cell activation and proliferation, and may promote more durable benefits such as sustained responses and restoration or induction of T cell memory.

Doses of 50-125 mcg of AMV564 by CIV route, for example, 50 mcg, 75 mcg, 100 mcg doses, could maintain control of both MDSC and leukemic blasts. This dosing regimen could also be effective for MDS. In MDS patients with a lower blast burden, where MDSC likely play a more critical role in disease progression, an even lower dose may be effective (e.g. 5, 15, 30, 50 mcg by CIV route).

In solid tumor patients, administration of AMV564 at doses of 5-50 mcg, generating approximate steady state exposures ranging from 0.1-5 pM, can be effective in depleting MDSC and promoting a favorable CD4 and CD8 T cell activation profile and cytokine milieu to promote restoration of anti-tumor immunity. Higher doses of 50-75 mcg or 75-150 mcg would also yield exposures that remain within the selective range for MDSC depletion.

Circulating MDSC are a pharmacodynamic biomarker of AMV564 and T cell responses. As MDSC are known to be induced by T cell activation, they are induced as a consequence of the T cell activation stimulated by AMV564. The MDSC reflect engagement of AMV564 with target cells (MDSC) and depletion of such cells, and in relationship to dose for a bivalent, bispecific T cell engager such as AMV564, they reflect effective dosing within the optimal therapeutic index of the drug, to enable effective depletion of these comparatively rare cells as compared to the rest of the CD33 positive myeloid lineage.

Because AMV564 depletes MDSC, treatment with AMV564, for example, under the dosing regimens described herein, can be useful for depleting MDSC in a variety of contexts. For example, AMV564 can be used to deplete MDSC in patients being treated with a therapy that activates T cells or involves administration of activated T cells.

Management of Cytokine Release Syndrome (CRS)

CRS, while not fully understood, appears to be related to T cell activation and subsequent activation of macrophages and other myeloid cells to generate and secrete IL-6, IL-1B and other cytokines. CRS is commonly associated with T cell engaging therapies such as T cell engaging bispecific antibodies and CAR-T therapy. CRS is most apparent at initiation of dosing. As shown below, administration of AMV564 by subcutaneous route in solid tumor patients at doses, for example, of 15-50 mcg results in robust T cell activation as assessed by various metrics including up to 10-40× increases from baseline in detectable peripheral Interferon gamma (IFNγ) in the first cycle of dosing. However, the increase in IL-6 is comparatively modest (the two cytokines are around 1:1 or favor higher IFNγ) (see FIGS. 7A-7E) and IL-113 is not detected at significant levels. This favorable profile is consistent with the absence of CRS observed in patients in this clinical study. This favorable profile of demonstrably strong T cell activation with lack of CRS could reflect a combination of features including the depletion of MDSC (which can produce inflammatory cytokines), bivalent T cell engagement by AMV564 (which may more closely resemble a more native T cell receptor engagement) and the lymphatic delivery and distribution kinetics associated with subcutaneous injection of AMV564. AMV564 has a favorable therapeutic index that is amenable to chronic dosing, these properties should also assist in the mitigation of CRS after lead in dosing to target dose is completed.

Combination Therapies

Effective treatment of tumorigenesis is often achieved via combination therapy. The functional therapeutic index of AMV564, with a dosage range that maximizes both efficacy and safety (notably lack of significant depletion of normal myeloid cells) positions it well for combination therapy approaches. Combination strategies include but are not limited to, in solid tumors, checkpoint blockade (PD-1 or PDL-1 blocking agents), T cell activators and expanders such as cytokines IL-2, IL-10 and IL-15, dual targeting agents such as those targeting checkpoint (e.g. PD-1 or PDL-1) and immune repression (e.g. TGFβ), CAR-T therapies, NK activating therapies, or standard of care chemotherapy. In AML and MDS, the previously listed therapies could also be used in combination, along with other established agents in AML such as hypomethylating agents (e.g. azacytidine, decitabine), differentiation agents (e.g. targeting IDH1/2), targeted agents (e.g. against FLT3), agents targeting anti-apoptotic proteins such as BCL2 (e.g. venetoclax), BCL-XL, or MCL1, or lenalidomide.

AMV564 can be used alone or in combination to treat melanoma (e.g., patients with unresectable or metastatic melanoma, melanoma with involvement of lymph node(s) following complete resection); non-Small Cell Lung Cancer (NSCLC) (e.g., metastatic non-squamous NSCLC, III NSCLC, metastatic NSCLC expressing PD-L1); head and Neck Squamous Cell Cancer (HNSCC); Classical Hodgkin Lymphoma (cHL); Primary Mediastinal Large B-Cell Lymphoma (PMBCL); urothelial Carcinoma (e.g., locally advanced or metastatic urothelial carcinoma expressing PD-L1); Microsatellite Instability-High Cancer (e.g., unresectable or metastatic, microsatellite instability-high (MSI-H) or mismatch repair deficient; solid tumors that have progressed following prior treatment; Gastric Cancer (e.g., recurrent locally advanced or metastatic gastric or gastroesophageal junction adenocarcinoma expressing PD-L1); Cervical Cancer; Hepatocellular Carcinoma (HCC); Merkel Cell Carcinoma (MCC); and Renal Cell Carcinoma (RCC).

### BRIEF DESCRIPTION OF THE FIGURES

FIGS. 1A-1G present data showing that AMV264 depletes M DSC and activates T cells ex vivo and that AMD564 binds MSC and KG-1 cells at 1 and 10 pm, but does not bind monocytes at these concentrations.

FIGS. 2A-2F present data showing that that AMV264 depletes MDSC and activates T cells in patients. The lighter bar indicates the days of the lead in dose and the darker bar indicates the days of the target dose.

FIGS. 3A-3C present data showing the impact of AMV264 on peripheral blood MDSC, bone marrow MDSC, peripheral blood T cells. The lighter bar indicates the days of the lead in dose and the darker bar indicates the days of the target dose.

FIGS. 4A-4D present data showing that AMV564 is a selective an potent conditional agonist. The single open circles and triangles show the result of a CD3/CD28 stimulation in the absence of AMV564.

FIGS. 5A-5D present data showing the MDSC control is associated with Treg control in solid tumor patients. The filled squared are G-MDSC and the filled circles are M-MDSC. The bars of the axis indicate AMV564 dosing days.

FIGS. 6A-6G present data showing that CD8:Treg ratio improves on AMV564 therapy in solid tumor patients The dotted line indicates the baseline ratio; bars along the x-axis indicate dosing days and the broad bar indicates the ratio for healthy controls.

FIGS. 7A-7E present data showing that AMV564 promotes favorable CD4 and CD8 T cell polarization in an ovarian cancer patient. The dotted line indicates the baseline ratio; bars along the x-axis indicate dosing days and the broad bar indicates the ratio for healthy controls.

FIGS. 8A-8F show IFNγ Cycle 1, IFNγ Cycle 2, IL-6 Cycle 1 and IL-6 Cycle 2 levels in six solid tumor patients treated with AMV564.

FIGS. 9A-9D show the results of M-MDSC and G-MDSC measurements in four solid tumor patients treated with AMV564 in combination pembrolizumab. The filled squared are G-MDSC and the filled circles are M-MDSC. The bars of the axis indicate AMV564 dosing days.

FIGS. 10A-10D show the impact of AMV564 in combination pembrolizumab on T-Bet and granzyme B positive CD8 cells and CD8/Treg ratio in two solid tumor patients. The dotted line indicates the baseline ratio; bars along the x-axis indicate AMV564 dosing days and the broad bar indicates the ratio for healthy controls.

FIGS. 11A-11B show the impact of AMV564 in combination pembrolizumab treat on CD8 cell proliferation and activation in two solid tumor patients.

### DETAILED DESCRIPTION

AMV564

AMV564 is a homodimer of SEQ ID NO: 1. AMV564 is described in U.S. Pat. No. 9,212,225 (Diabody 16; SEQ ID NO: 113 without the 6 His tag at the amino terminus) and WO 2016/196230 (SEQ ID NO:139). A pharmaceutical composition of AMV564 comprises a polypeptide having the amino acid sequence of SEQ ID NO:1 and a pharmaceutically acceptable carrier or excipient.

AMV564
(SEQ ID NO: 1)
DIQMTQSPSS LSASVGDRVT ITCRSSTGAV TTSNYANWVQ
QKPGKAPKAL IGGTNKRAPG VPSRFSGSLI GDKATLTISS
LQPEDFATYY CALWYSNLWV FGQGTKVEIK GGSGGSQVQL
VQSGAEVKKP GASVKVSCKA SGYTFTSYDI NWVRQAPGQG
LEWMGWMNPN SGNTGFAQKF QGRVTMTRDT STSTVYMELS
SLRSEDTAVY YCARDRANTD YSLGMDVWGQ GTLVTVSSGG
SGQSVLTQPP SASGTPGQRV TISCSGSRSN IGSNTVNWYQ
QLPGTAPKLL IYGNNQRPSG VPDRFSGSKS GTSASLAISG
LQSEDEADYY CATWDDSLIG WVFGGGTKLT VLGGSGGSEV
QLVESGGGLV QPGGSLRLSC AASGFTFSTY AMNWVRQAPG
KGLEWVGRIR SKYNNYATYY ADSVKDRFTI SRDDSKNSLY
LQMNSLKTED TAVYYCARHG NFGNSYVSYF AYWGQGTLVT VSS

### Example 1: AMV564 Depletes MDSC and Activates T Cells Ex Vivo

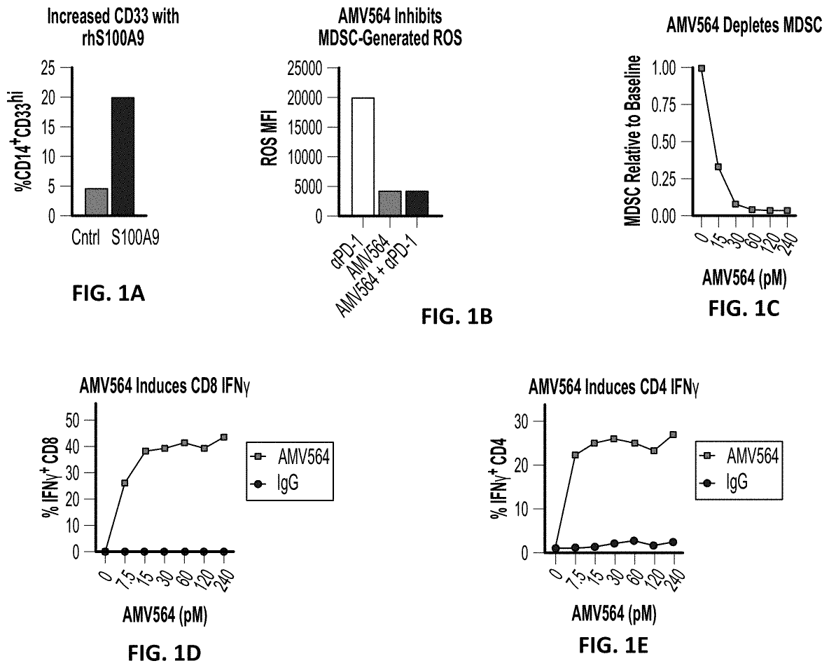

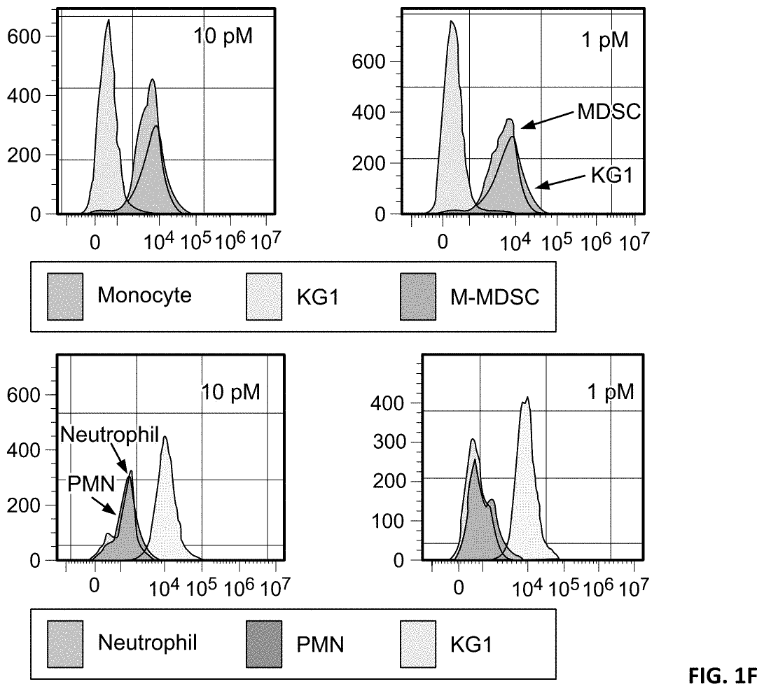

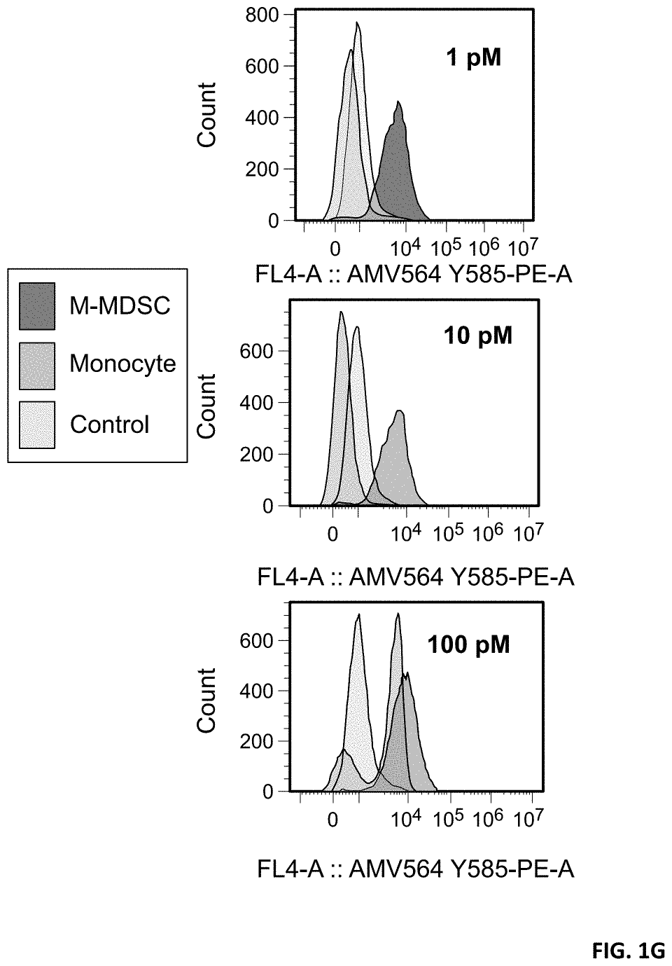

In this study it was found that AMV564 treatment of primary cells (PBMC, MDS bone marrow, tumor PBMC) ex vivo both depletes MDSC and actives T cells. FIG. 1A sows that treatment of PBMC with CD33 ligand S100A9 results in expansion of MDSC and increase in CD33 expression. In FIGS. 1B-1E it can be seen that exposure to AMV564: negates reactive oxygen species (ROS) produced in response to S100A9 stimulation of PBMC to expand MDSC (FIG. 1B), causes selective depletion of MDSC (FIG. 1C), and increases in CD8 T cell (FIG. 1D) and CD4 T cell (FIG. 1E) numbers and activation state (as assessed by IFNγ positive fraction).

Thus, treatment ex vivo of patient-derived peripheral blood mononuclear cells (PBMC) resulted in selective depletion of MDSCs (p<0.01) and a decrease in the production of reactive oxygen species. AMV564 induced a significant increase in activated T cells only in the presence of CD33+ target cells, with >2-fold increase in the proliferation of CD4+ and CD8+ T cells. The increase in proliferation was dose-dependent and accompanied by a significant increase in IFNγ production.

As showing in FIG. 1F, AMV564 binds to MDSC (and leukemic blast line KG1) at 1 and 10 pM. However, at these concentrations, there is essentially no evidence of binding to monocytes, neutrophils and polymorphonuclear leukocytes (PMN). These concentrations are within the range of exposure observed for dosing of AMV564 by subcutaneous route at doses of 5-15-50 mcg (about 0.1-5 pM). As shown in FIG. 1G.

### Example 2: AMV564 Depletes MDSC and Activates T Cells in Patients

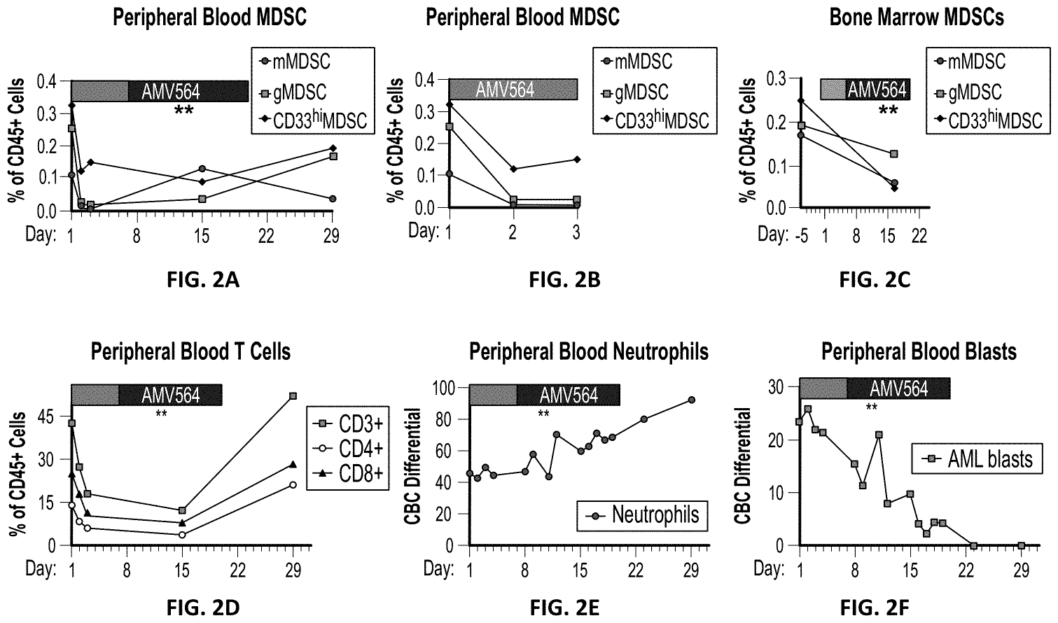

In this clinical study, it was found that AMV564 treatment leads to depletion of peripheral blood MDSC and bone marrow MDSC. The results of this analysis are shown in FIGS. 2A-2F where it can also be seen that AMV564 treatment resulted in depletion of AML blasts with no decrease in neutrophils. As can be seen, rapid depletion of both monocytic and granulocytic MDSCs is apparent with little or no impact on circulating neutrophil or monocytes populations. Evidence of early T cell activation is apparent with rapid re-distribution/margination of T cells (this apparent transient lymphopenia is a consequence of T cell activation and migration to lymph nodes and tissues). In FIGS. 2A-2F the period of lead-in AMV564 dosing (15 mcg/kg; 3 days) is indicated by the lighter colored bar and the target AMV564 dosing (100 mcg/kg) is indicated by the darker colored bar). In FIGS. 2A and 2B it can be seen the peripheral blood MDSC are depleted. In FIG. 2C it can be seen the bone marrow MDSC are depleted. FIGS. 2D-2E, respectively, show the impact of AMV564 treatment on peripheral blood T cells, peripheral blood neutrophils, and peripheral blood blasts.

### Example 3: AMV564 Depletes MDSC in Solid Tumor Patients

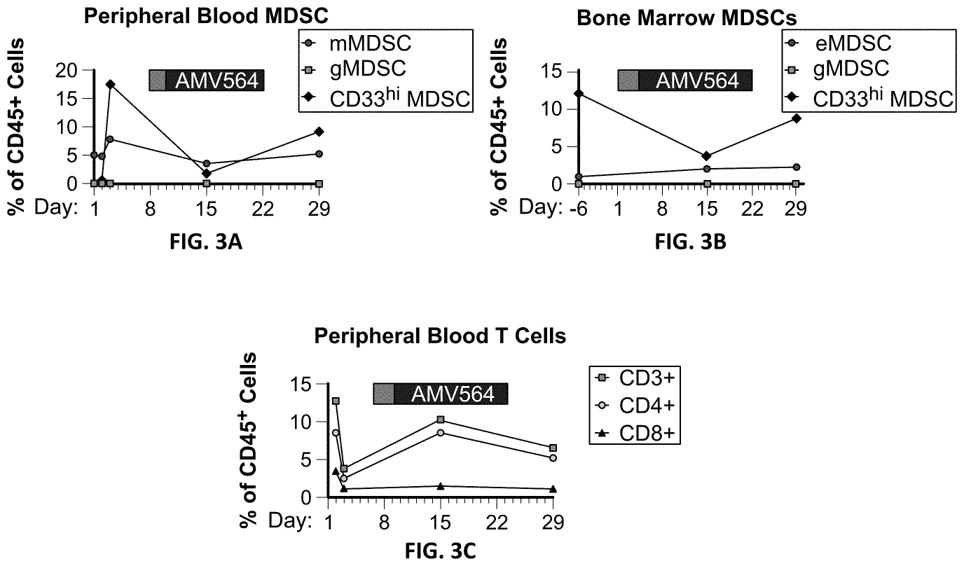

As shown in FIGS. 3A-C, an initial increase in peripheral blood MDSC in response to T cell activation is observed in lead in dosing (Days 1-3 in some patients). FIG. 3C shows the rapid redistribution/margination of peripheral blood T cells, consistent with T cell activation, as T cells transmigrate to lymph nodes and tissues. However, at the target dose, peripheral MDSC are controlled. Bone marrow MDSC are also substantially decreased when assessed at day 15 relative to baseline. However, both bone marrow and peripheral blood MDSC can rebound once AMV564 treatment is stopped.

### Example 4: AMV564 is a Selective and Potent Conditional Agonist

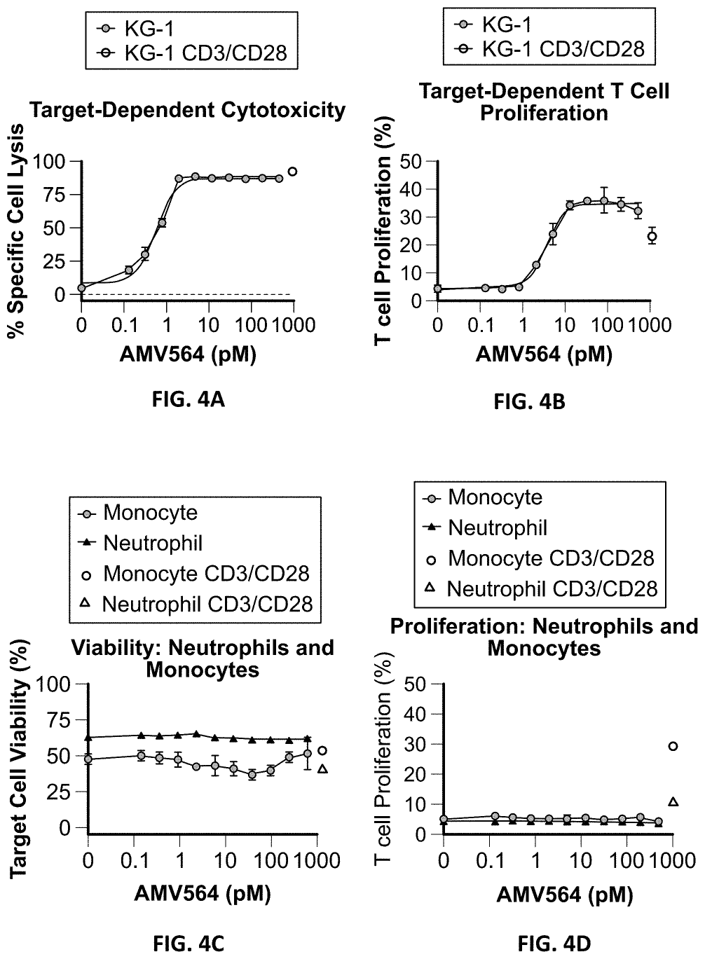

Primary human T cells and KG-1 cells were exposed to AMV564. Target-dependent cytotoxicity (FIG. 4A), target dependent T cell proliferation (FIG. 4B), viability of differentiated monocytes and neutrophils (FIG. 4C), viability of differentiated monocytes and neutrophils (FIG. 4D) were measured, all with CD3/CD28 used as reference T cell stimulation. As KG1 expresses CD33 and AMV564 shows similar binding to KG1 as it does to MDSC, KG1 was used as a surrogate for MDSC in these assays. As can be seen in FIGS. 4A-4D, AMV564 induces potent dose-dependent cell death of KG1 (FIG. 4A), at a maximum level similar to a CD3-CD28 stimulation. This is accompanied by an increase in daughter cells, reflective of T cell proliferation (FIG. 4B) at levels equivalent to or exceeding the CD3-CD28 reference stimulation. However, there is no evidence of AMV564 promoting significant cell death for autologous monocytes or neutrophils (FIG. 4C), and similarly, no evidence of any induction of T cell proliferation (FIG. 4D) with these cell populations, unlike general T cell stimulation using CD3-CD28.

### Example 5: Phase 1 Clinical Study of AMV564 in Patients with Solid Tumors

This study enrolled adult patients having non-resectable, advanced metastatic solid tumors that are recurrent and progressing since the last anti-tumor therapy and for which no recognized standard therapy exists. The patients had ECOG performance status of and adequate organ function. Patient were treated with AMV564 alone (15, 50, or 75 mcg/day) or AMV564 (5, 15, or 50 mcg/day) in combination Pembrolizumab administered intravenously at 200 mg every 3 week (Q3W). In both cases, AMV564 was administered once a day by subcutaneous injection on days 1-5 and days 8-12 of a 21-day cycle. AMV564 was well tolerated and pharmacodynamic analyses showed evidence of relief of immune suppression (decreased MDSC and Treg) and promotion of effector CD8 and Th1 CD4 responses in this Phase 1 population of heterogeneous cancer patients previously treated with other therapies. In general, patients treated with AMV564 exhibited a cytokine profile consistent with activation of T cells including CD4 Th1 helper cells, antigen presenting cells, and improved T cell trafficking to tissues such as tumor tissues (increase in IFNγ, IL-15, IL-18, soluble granzyme B and CXCL10). While strong T cell activation was observed, there were no episodes of cytokine release syndrome.

### Example 6: MDSC Control is Associated with Treg Control in Solid Tumor Patients

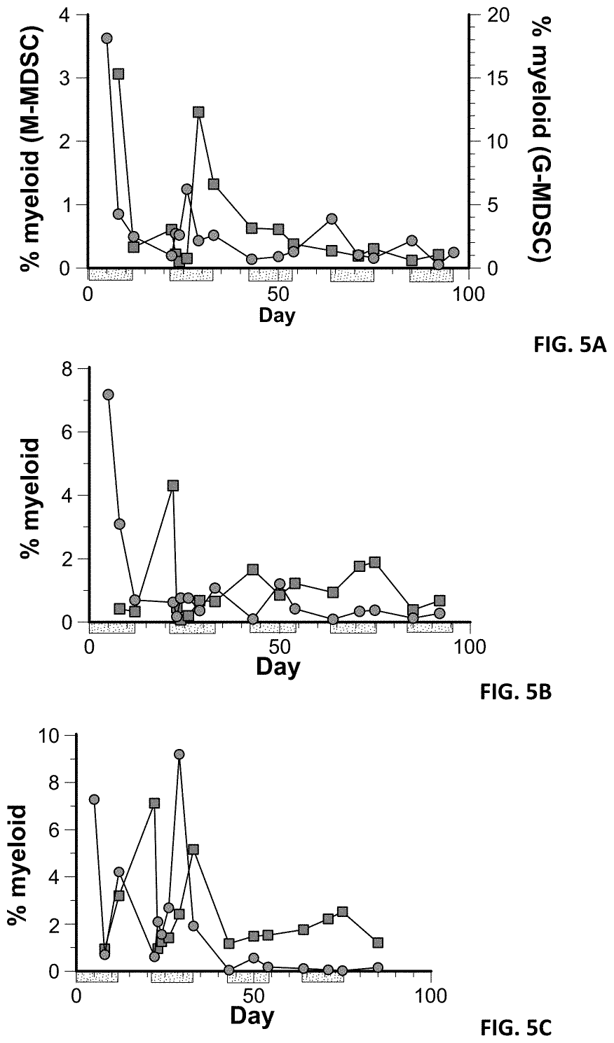

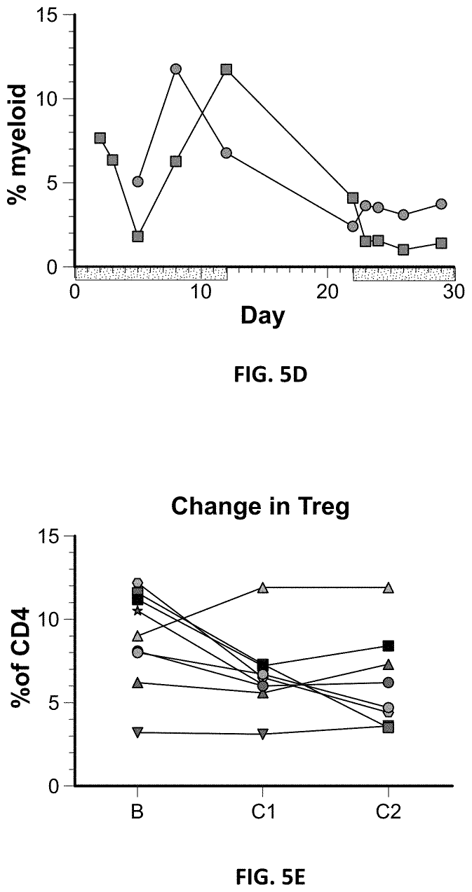

FIGS. 5A-5D show that M-MDSC and G-MDSC are controlled in patients with solid tumors (FIG. 5A: ovarian—15 mcg AMV564; FIG. 5B: cutaneous 50 mcg AMV564; FIG. 5C: small bowel—15 mcg AMV564; FIG. 5D: gastroesohageal junction—15 mcg AMV564) that were treated with AMV564 (once a day by subcutaneous injection on days 1-5 and days 8-12 of a 21-day cycle). FIG. 5E depicts the change in Treg from baseline (B) through two cycles (C1 and C2) of therapy (squares=ovarian cancer; pink circles=small bowel; green circles=cutaneous carcinoma; upper triangles=gastroesohageal junction).

### Example 7: CD8:Treg Ratio Improves on AMV564 Therapy in Solid Tumor Patients

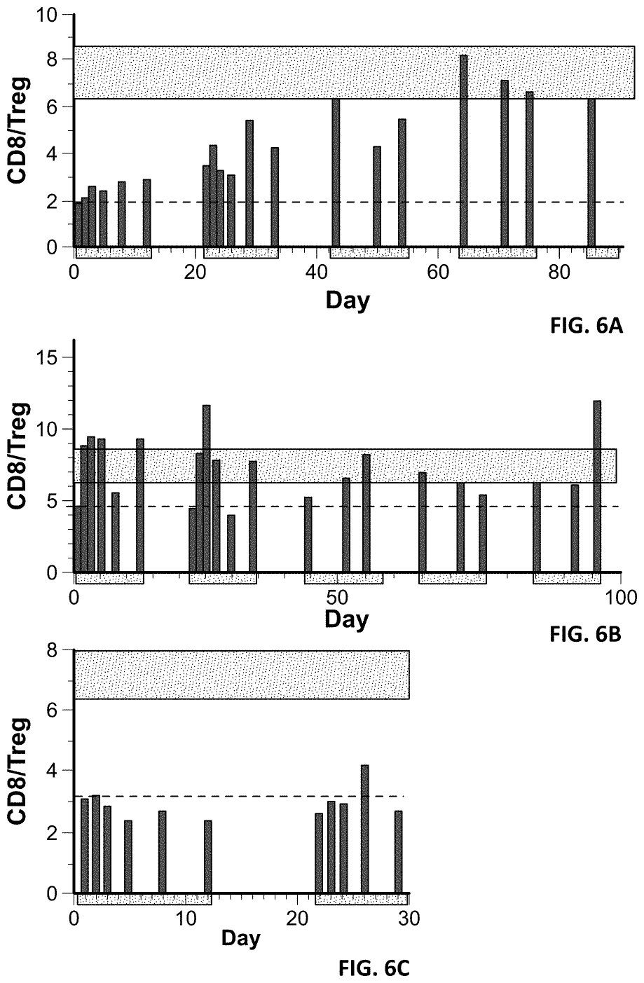

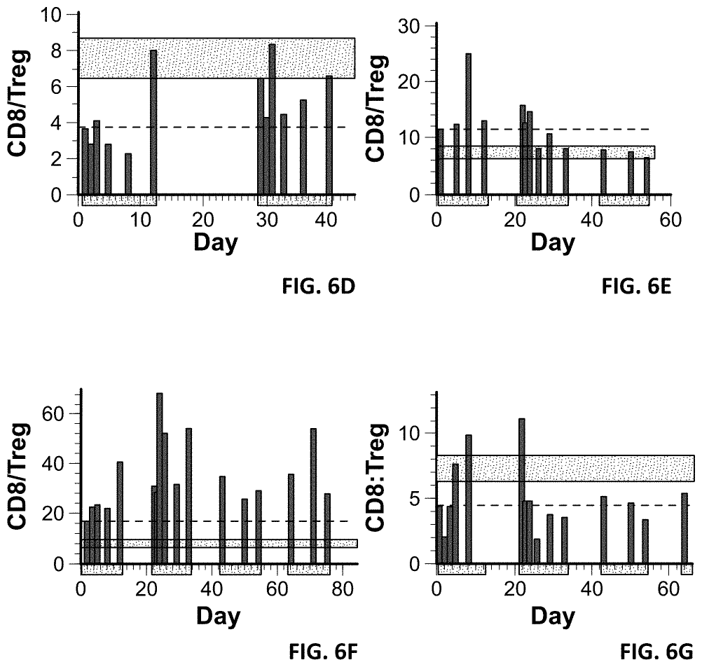

FIGS. 6A-6G show that there an increase in the CD8/Treg ratio (for most subjects) while solid tumor patients (FIG. 6A: Small Bowel—Stable Disease; FIG. 6B: Ovarian—Complete Response; FIG. 6C: GE Junction—Progressive Disease; FIG. 6D: Endometrial—Stable Disease; FIG. 6E: Colorectal—Progressive Disease; FIG. 6F: Cutaneous—Stable Disease; FIG. 6G: Appendiceal—Stable Disease) were on AMV564 therapy (once a day by subcutaneous injection on days 1-5 and days 8-12 of a 21-day cycle (FIGS. 6A-6C 15 mcg; FIG. 6D-6G 50 mcg). The dotted line indicates the baseline ratio; bars along the x-axis indicate dosing days and the broad bar indicates the ratio for healthy controls whose peripheral blood samples were processed in the same flow based assay.

### Example 8: AMV564 Promotes Favorable CD4 and CD8 T Cell Polarization in an Ovarian Cancer Patient

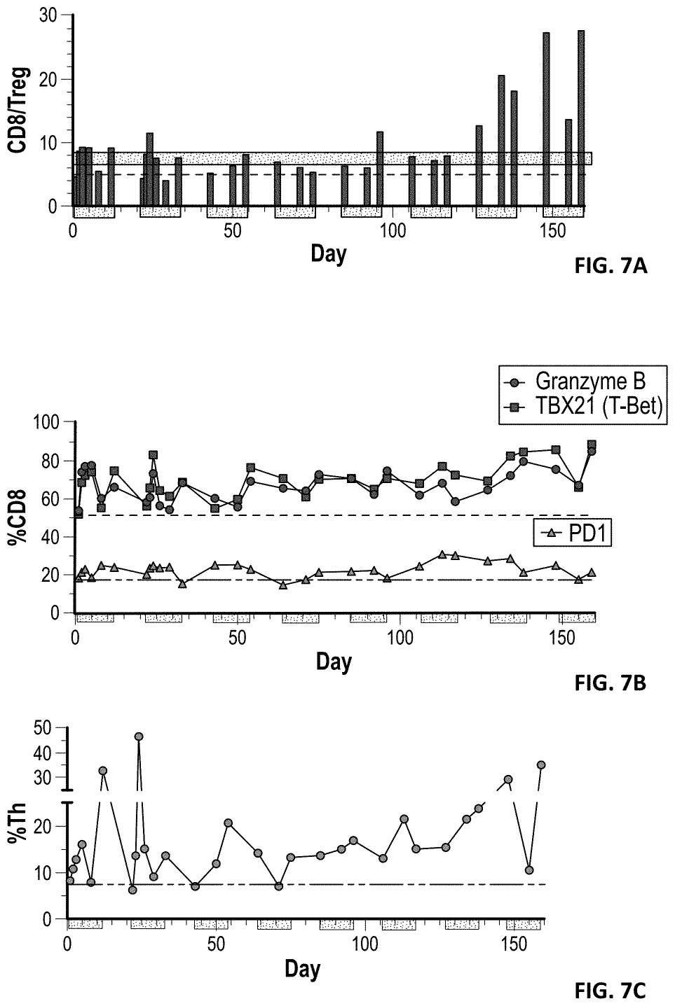

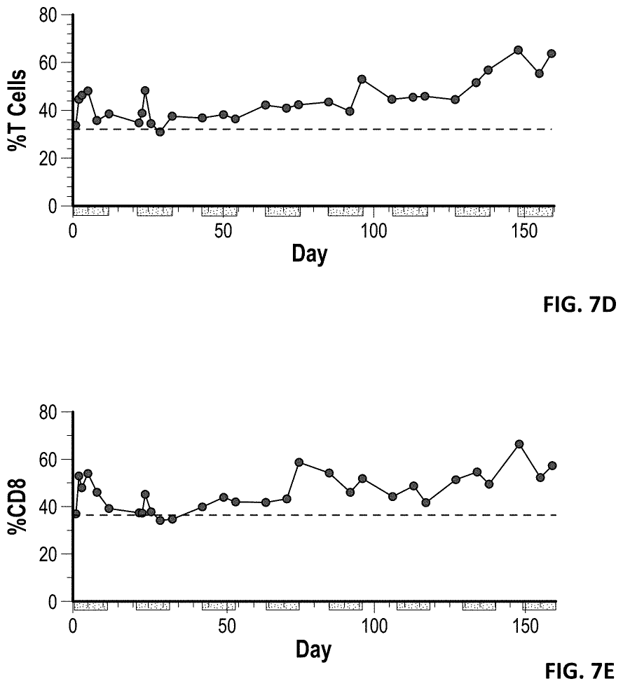

An ovarian cancer patient who had previously undergone multiple lines of platinum-based chemotherapy, surgery, radiation, pembrolizumab (best response stable disease, completed 6 months prior to study start), and therapy with niraparib and letrozole, was treated with 15 mcg AMV564 (once a day by subcutaneous injection on days 1-5 and days 8-12 of a 21-day cycle). This patient exhibited an ongoing increase in CD8/Treg, increase in % CD8, maintained or increased effector CD8 (TBX21 and/or granzyme B positive) and memory CD8 cells, a dynamic increases in TBX21-positive CD4 T helper cells and dynamic modulation of PD1 positive CD8 fraction but without substantial overall increase as shown in FIGS. 7A-7E. This patient progressed from stable disease, to partial response to complete response as assessed by CT scans at 6-8 week intervals.

### Example 9: Patients Treated with AMV564 Exhibit Signs of T Cell Activation without CRS

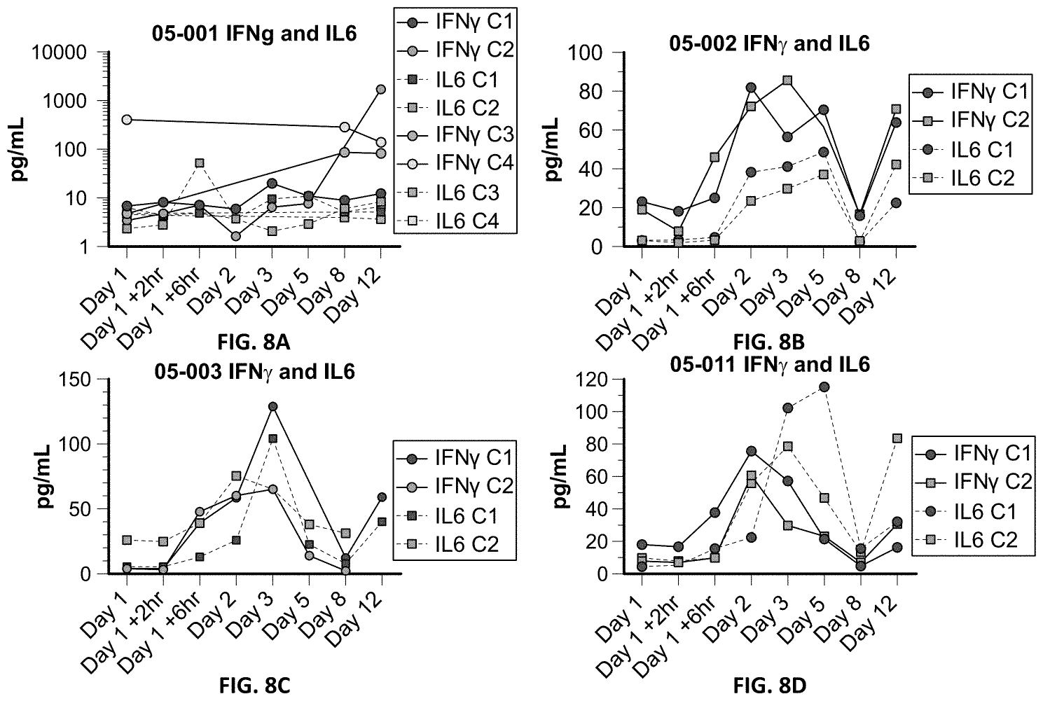

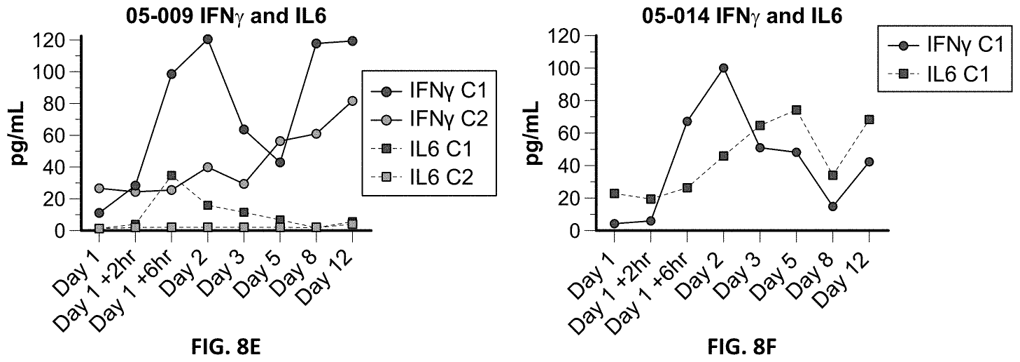

FIGS. 8A-8F (Patients 1, 2, 3, 11, 9 and 14, respectively) show the results of IFNγ and IL-6 measurements in six solid tumor patients treated with AMV564 (once a day by subcutaneous injection on days 1-5 and days 8-12 of a 21-day cycle). As can be seen there is clear evidence of systemic IFNγ production without excessive IL-6 production (ratio of IFNγ: IL-6 was about 1:1 or better for most patients).

Over 5 cycles of treatment, a solid tumor patient (data not shown) exhibited increases in IFNγ, TNFα and IL18 and other factors at later time points (e.g., end of cycle 2 and cycles 3, 4) consistent with MHC class I upregulation, dendritic cell activation and T cell trafficking (decrease in MDSC-inducing factor G-CSF). This same patient entered the study with poor CD8/Treg at baseline. Improvement was observed throughout treatment, particularly around cycle 3, where increases in CD8 T cell proliferation and activation (Ki67 and CD38 fractions) were observed, consistent with improvement in CD8 effector function (T-bet and granzyme B positive fractions). This mirrors timing of observations for increases in cytokines and factors consistent with improved dendritic cell activation and Th1 response, suggesting that AMV564 was, over time, driving a more favorable immune polarization even in this late-stage patient.

### Example 10: Treatment with AMV564 and Pembrolizumab

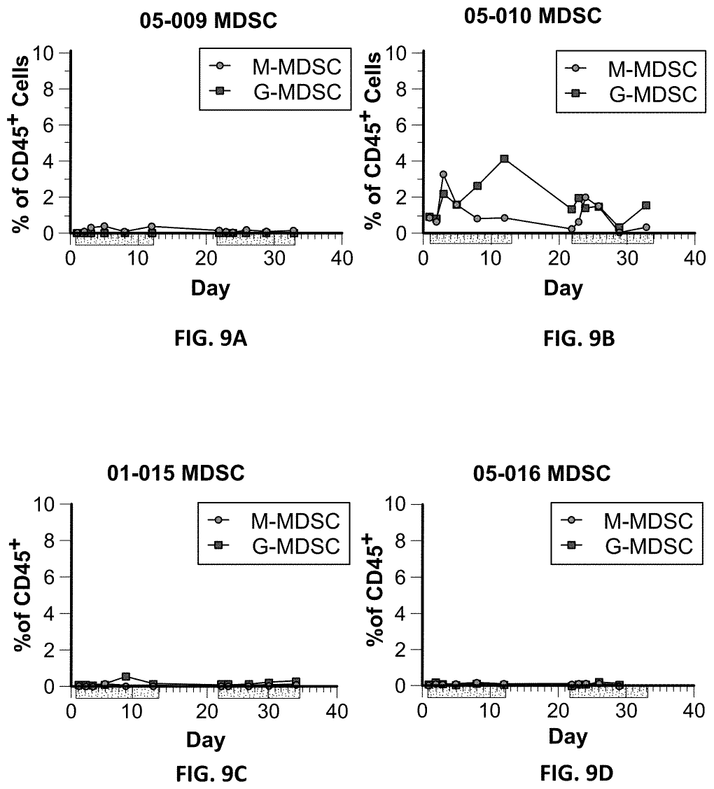

FIGS. 9A-9D show the results of M-MDSC and G-MDSC measurements in four solid tumor patients treated with AMV564 once a day by subcutaneous injection on days 1-5 and days 8-12 of a 21-day cycle (5 mcg/day (FIG. 9A and FIG. 9B) or 15 mcg/day (FIG. 9C and FIG. 9D)) in combination pembrolizumab administered intravenously at 200 mg every 3 week (Q3W). The AMV564 administration days are indicated by a bar along the x-axis and the pembrolizumab treatment days are indicated with an asterisk. As can be seen, very good MDSC control was observed.

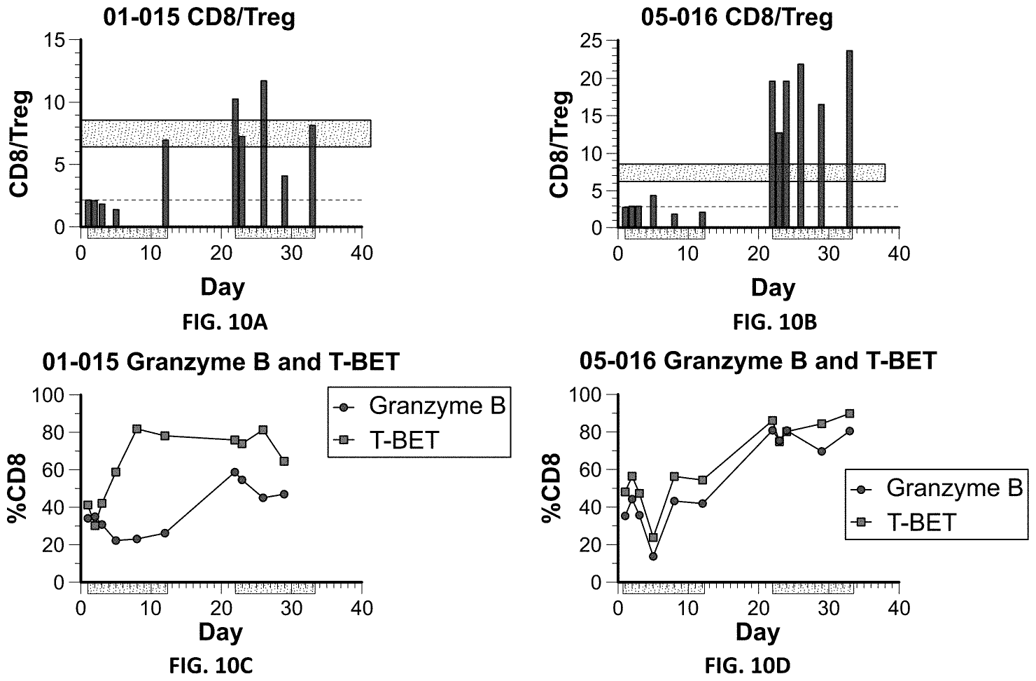

FIGS. 10A-10D shows data from two patients (FIG. 10A and FIG. 10C: Patient 15; FIG. 1013 and FIG. 10D: Patient 16) treated with AMV 564 (15 mcg once a day by subcutaneous injection on days 1-5 and days 8-12 of a 21-day cycle in combination pembrolizumab (administered intravenously at 200 mg Q3W). This data shows evidence of a substantial increase in CD8 effector cell fraction in cycles 1-2 and a substantial increase in CD8/Treg ratio. The data also show expansion of T-Bet and granzyme B positive CD8 cells. These effects were not apparent in 5 mcg AMV564 combination cohort in this study.

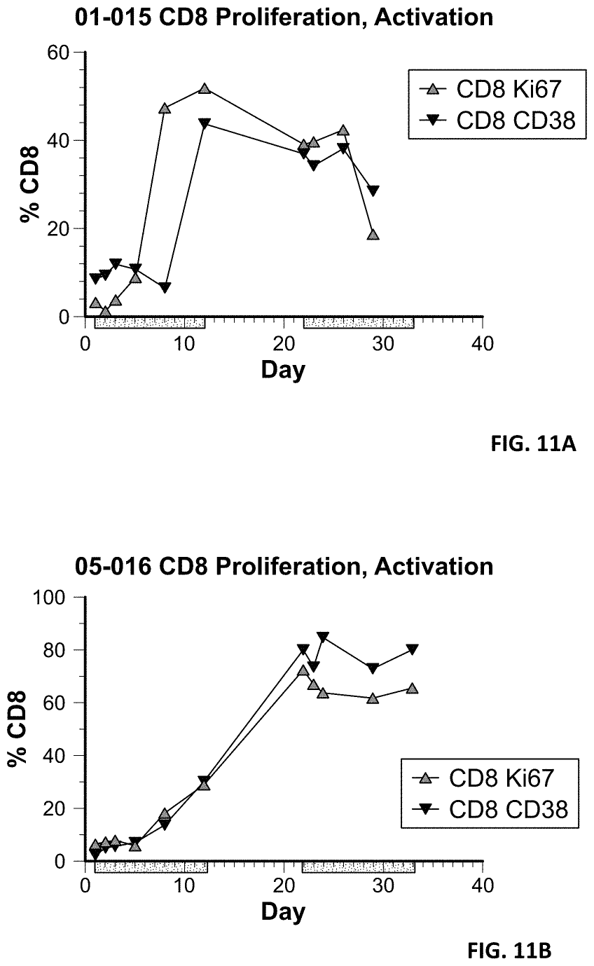

FIGS. 11A-11B shows data from two patients (FIG. 11A: Patient 15; FIG. 11B: Patient 16) treated with AMV 564 (15 mcg once a day by subcutaneous injection on days 1-5 and days 8-12 of a 21-day cycle in combination pembrolizumab (administered intravenously at 200 mg Q3W). This data shows evidence of significant increase in CD8 proliferation (assessed by CD8 Ki67) and activation (assessed by CD 8 CD38). The substantial and rapid increases in 2 of 3 patients dosed in combination, from a poor baseline level, suggests potential combination benefit if AMV564 and pembrolizumab.

1. A method for reducing myeloid-derived suppressor cells and activating T cells in a patient, the method comprising administering AMV564 to the patient.

2.-43. (canceled)


SEQUENCE LISTING (1 sequence)

SEQ ID NO: 1 - protein, 483 aa, Artificial Sequence
  Polypeptide containing scFv domains
DIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGSGGSQVQLVQSGAEVKKPGASVKVSCKASGYTFTSYDINWVRQAPGQGLEWMGWMNPNSGNTGFAQKFQGRVTMTRDTSTSTVYMELSSLRSEDTAVYYCARDRANTDYSLGMDVWGQGTLVTVSSGGSGQSVLTQPPSASGTPGQRVTISCSGSRSNIGSNTVNWYQQLPGTAPKLLIYGNNQRPSGVPDRFSGSKSGTSASLAISGLQSEDEADYYCATWDDSLIGWVFGGGTKLTVLGGSGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSS